(19) United States
(12) Patent Application Publication
(10) Pub. No.: US 2025/0242072 A1
(43) Pub. Date: Jul. 31, 2025

(54) ULTRAVIOLET FILTER DEVICE AND RELATED METHODS THEREOF

(71) Applicant: University of Virginia Patent Foundation, Charlottesville, VA (US)

(72) Inventor: Robert H. Thiele, Charlottesville, VA (US)

(21) Appl. No.: 19/037,940

(22) Filed: Jan. 27, 2025

Related U.S. Application Data
(60) Provisional application No. 63/625,443, filed on Jan. 26, 2024.

(51) Int. Cl. A61L 2/26; A61L 2/10; A61L 2/24

(52) U.S. Cl. CPC A61L 2/26; A61L 2/10; A61L 2/24; A61L 2202/11; A61L 2202/24

(57) ABSTRACT

Described herein are devices, systems, kits, and methods for, among other things, providing UV-C irradiation to central lines that are utilized to administer aqueous therapeutics that are delivered intravenously to a subject.

### CROSS REFERENCE TO RELATED APPLICATION[S]

This application claims priority to, and the benefit of, U.S. Provisional Patent Application Ser. No. 63/625,443 filed on Jan. 26, 2024, which is incorporated herein by reference in its entirety.

### BACKGROUND

Central line-associated blood-stream infections (CLABSI) are, in aggregate, the second most costly infectious problem in the United States. Such infections can cost up to $48,000 per incident. The use of disposal hardware combined with traditional sterilization and aseptic techniques, while helpful, are not sufficient to mitigate the financial cost or patient outcome of such infections. Accordingly, there is a need to address the aforementioned deficiencies and inadequacies in order to better address the problems and costs associated with CLABSI.

### SUMMARY

Described herein are systems, methods, computer readable media, and methods relating to the sterilization of fluids for delivery into a patient, in particular, through a central line.

Described herein are sterilization cartridges. In embodiments, a sterilization cartridge comprises a housing having a fluidic conduit, an inflow channel, and an outflow channel. In embodiments, the fluidic conduit, inflow channel, and outflow are in fluidic communication. In embodiments, a side of the housing is translucent and configured to expose the fluidic conduit to a light source. In embodiments, the other sides of the housing can be opaque (i.e., configured so they do not pass UV light) or can also be translucent. Embodiments of sterilization cartridges can further comprise an inflow adaptor in fluidic communication with the inflow channel configured to removably couple to intravenous tubing. Embodiments of sterilization cartridges can further comprise an outflow adaptor in fluidic communication with the outflow channel configured to removably couple to intravenous tubing.

In embodiments, the housing does not have the inflow and outflow adaptors that allow for the removable connection between the housing and the tubing, and the housing and the inflow tubing and/or outflow tubing are non-removably attached to each other (and in fluidic communication) permanently in a manner that would require cutting the tubing with a cutting device in order to make it removable.

Embodiments of sterilization cartridges can further comprise a fluid mixing device. In embodiments, the fluid mixing device is in the fluidic conduit.

In embodiments of sterilization cartridges described herein, a cartridge can further comprise a fluid mixing device comprising one or more baffling structures in the fluidic conduit.

In embodiments of sterilization cartridges described herein, a cartridge can further comprise a fluid restriction device in fluidic communication with the outflow channel.

In embodiments of sterilization cartridges described herein, the translucent side of the housing comprises UV-translucent polymer or quartz glass. In embodiments of sterilization cartridges described herein, the UV-translucent polymer comprises TOPAS® 8007 X-10 polymer.

In embodiments of sterilization cartridges described herein, the housing can further comprise one or more ultraviolet light (UV) sources.

In embodiments of sterilization cartridges described herein, the housing can further comprise a flowmeter. In embodiments, the flowmeter may comprise one or more photodiodes. In embodiments of sterilization cartridges described herein, the housing can further comprise one or more ultraviolet light (UV) sources and a flowmeter.

Embodiments of sterilization cartridges can further comprise one or more central processing units configured to send and receive data from the flowmeter and the one or more UV light sources, wherein the data sent to flowmeter, one or more UV light sources, or both, is based on a flow rate of a liquid through the cartridge determined by the flowmeter.

Embodiments of sterilization cartridges can further comprise one or more indicator lights coupled to an exterior surface of the housing.

In embodiments of sterilization cartridges described herein, the housing further comprises a photodiode.

In embodiments of sterilization cartridges described herein, the housing can be configured to be reusable across different users.

Embodiments of sterilization cartridges can further comprise a power source.

In embodiments of sterilization cartridges described herein, the power source comprises a battery or one or more insulated wires running axially through an intravenous tubing in fluidic connection with the inflow adaptor.

In embodiments of sterilization cartridges described herein, the inflow adaptor and outflow adaptor each comprise a Luer (ISO 80369-7) connection.

Described herein are kits for sterilizing fluids to be administered to a patient. In embodiments, a kit can comprise a sterilization cartridge as described herein and one or more intravenous tubings. In embodiments, kits can comprise instructions for use. In embodiments of kits according to the present disclosure, at least one of the one or more intravenous tubings comprises one or more insulated wires extending axially along an exterior surface or through walls of the intravenous tubing configured to provide power to the one or more ultraviolet light (UV) sources, the flowmeter, or both. In embodiments of kits according to the present disclosure, a kit can comprise a catheter and an introducer needle.

Described herein are systems for sterilizing fluids to be administered to a patient. In embodiments, a system for sterilizing fluids in intravenous tubing comprises a sterilization cartridge as described herein, one or more intravenous tubings configured to connect to the inflow adaptor, one or more intravenous tubings configured to connect to the outflow adaptor, and one or more ultraviolet light (UV) sources. In embodiments of systems described herein, the sterilization cartridge comprises a fluid restriction device in fluidic communication with the outflow channel.

In embodiments of systems described herein, the housing further comprises one or more baffling structures in the fluidic conduit.

In embodiments of systems described herein, the translucent side of the housing comprises UV-translucent polymer or quartz glass.

In embodiments of systems described herein, the UV-translucent polymer comprises TOPAS® 8007 X-10 polymer.

In embodiments, systems further comprise a flowmeter. In embodiments, systems further comprise an infusion pump.

In embodiments, systems further comprise one or more central processing units configured to send and receive data from the flowmeter and the one or more UV light sources, wherein the data sent to flowmeter, one or more UV light sources, or both, is based on a flow rate of a liquid through the cartridge determined by the flowmeter.

In embodiments, systems further comprise one or more indicator lights coupled to an exterior surface of the housing.

In embodiments of systems described herein, the housing further comprises a photodiode.

In embodiments of systems described herein, the housing is configured to be reusable across different users.

In embodiments of systems described herein, systems further comprise a power source. In embodiments of systems described herein, the power source comprises a battery or one or more insulated wires running axially through at least one of the one or more intravenous tubings connected to the inflow adaptor.

In embodiments of systems described herein, the inflow adaptor and outflow adaptor each comprise a Luer (ISO 80369-7) connection.

In embodiments of systems described herein, systems further comprise one or more intravenous fluids, one or more vasoactive drugs, one or more antibiotics, one or more blood thinners, one or more sedating agents, one or more paralyzing agents, insulin, or one or more blood products in a pharmaceutically-acceptable fluidic vehicle. In embodiments of systems described herein, the one or more vasoactive drugs comprise insulin, epinephrine, Digoxin, Labetalol, or Amiodarone.

Described herein are methods of sterilizing fluids for delivery into a subject. In embodiments, described herein is a method of sterilizing fluids for delivery into a subject, comprising providing a fluidic path for delivery of a fluid to a subject by connecting the inflow adaptor of a sterilization cartridge described herein to intravenous inflow tubing, connecting the outflow adaptor of a sterilization cartridge described herein to intravenous outflow tubing, and connecting the intravenous outflow tubing into a vein of the subject. Methods further can comprise directing a fluid through the intravenous tubing into the intravenous tubing sterilization cartridge. Methods further can comprise applying ultraviolet (UV) light to the fluid as it passes through the fluidic conduit of the sterilization cartridge.

In embodiments of methods according to the present disclosure, methods can further comprise measuring an intensity of the UV light transmitted to the fluid with one or more photodiodes.

In embodiments of methods according to the present disclosure, methods can further comprise measuring a flow rate with a flowmeter, and further comprising adjusting the measured intensity of the UV light based at least in part on the measured flow rate of the fluid through the intravenous tubing sterilization cartridge.

In embodiments of methods according to the present disclosure, the fluid comprises one or more vasoactive drugs in a pharmaceutically-acceptable fluidic vehicle.

In embodiments of methods according to the present disclosure, the one or more vasoactive drugs comprise insulin, epinephrine, Digoxin, Labetalol, or Amiodarone.

In embodiments of methods according to the present disclosure, the subject has, or is suspected of having, diabetes, an adverse cardiac event, an adverse allergic event, low blood pressure, sepsis, a major surgery, a condition requiring total parenteral nutrition, a condition requiring intravenous antibiotics, immunosuppression, or a bone marrow transplant.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1 is an embodiment of a sterilization cartridge 100 according to the present disclosure.

FIGS. 2A and 2B depict additional embodiments and aspects of sterilization cartridges 100 according to the present disclosure.

FIGS. 3A and 3B are photographs depicting an application of sterilization cartridges, systems, and methods of the present disclosure. FIG. 3A is a photograph showing infusion tubing and intravenous catheter ports of which cartridges, systems, kits, and methods of the present disclosure can be utilized with. FIG. 3B depicts embodiments of placement of sterilization cartridges according to the present disclosure in-line with the intravenous catheter ports through which fluids, in particular fluids containing therapeutics, can be delivered to a subject in need thereof.

FIG. 4A is a perspective view of an embodiment of a sterilization cartridge 100 according to the present disclosure.

FIG. 4B is a top view of an embodiment of a sterilization cartridge 100 according to the present disclosure.

FIGS. 5A and 5B show an embodiment of aspects of a system 200 according to the present disclosure. Depicted is an embodiment of a heat sink and germicidal LED (i.e., a UV-C emitting LED) that can be utilized with sterilization cartridges according to the present disclosure. FIGS. 5A and 5B are opposing surfaces of the same embodiment.

FIGS. 6A and 6B show embodiments of the sterilization cartridge 100 of FIGS. 4A and 4B with inflow 109 and outflow adapters 111 that can be coupled to tubing, intravenous or central line tubing, for example.

FIG. 7 shows the effect of exposure of UV light (285 nm) on bacterial cultures over time (0, 1, 2, 5, and 10 seconds), in particular Meticillin-Sensitive Staphylococcus aureus (MSSA) (top panels) and Escherichia coli (E. coli).

FIG. 8 depicts an embodiment of a system 200 according to the present disclosure.

FIGS. 9A-9E show the effect of UVC light on vasoactive drug concentration over time. Plots of epinephrine (FIG. 9A), insulin (FIG. 9B), digoxin (FIG. 9C), labetalol (FIG. 9D), and amiodarone (FIG. 9E) are shown.

FIG. 10 is a plot showing the effect of UV-C irradiation on Staphylococcus aureus (S. aureas) and E. coli colony forming units. The initial CFU's tested were several log units greater than what may be practically present on a central line that could lead to CLABSI.

FIG. 11 shows the effect of UV-C irradiation on MSSA injected in the tail vein with and without UV-C irradiation. As can be seen, in non-irradiated lines, MSSA bacteria was found in liver 1105, spleen 1101, and kidney 1103 of the animals, as well as the catheter 1107 itself, wherein with irradiated lines, no bacteria was found in the organs of the animals or the catheter.

FIG. 12 is a is a block diagram illustrating an example of a machine which can be integrated with one or more aspects of embodiments of the present disclosure for implementation of devices, kits, systems, and methods as described herein.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of mechanical engineering, aseptic and sterilization, anesthetics, and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequences where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A device, system, kit, or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any device, system, kit, or method described as “comprising” (or which “comprises”) one or more named elements or steps also describes the corresponding, more limited composition or method “consisting essentially of” (or which “consists essentially of”) the same named elements or steps, meaning that the device, system, kit, or method includes the named essential elements or steps and can also include additional elements or steps that do not materially affect the basic and novel characteristic(s) of the device, system, kit, or method. It is also understood that any device, system, kit, or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any device, system, kit, or method disclosed herein, known or disclosed equivalents of any named essential element or step can be substituted for that element or step.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the terms “optional” or “optionally” means that the subsequently described event or circumstance can or cannot occur, and that the description includes instances where said event or circumstance occurs and instances where it does not.

As used herein, “individual”, “subject”, and “patient” refers to any living entity having or suspected of having: diabetes, an adverse cardiac event, an adverse allergic event, low blood pressure, sepsis, a major surgery, a condition requiring total parenteral nutrition, a condition requiring intravenous antibiotics, immunosuppression, or a bone marrow transplant.

A living organism can include mammals in particular, including a human being, and animals (e.g., vertebrates, amphibians, fish, mammals, cats, dogs, horses, pigs, cows, sheep, rodents, rabbits, squirrels, bears, primates (e.g., chimpanzees, gorillas)). These terms (“individual,” “subject,” “host,” and “patient”) used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.” In embodiments, the region of interest is any vein suitable for the introduction of a central line through with therapeutics can be administered. In embodiments, devices, systems, kits, and methods as described herein are only administered to humans.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

The term “blood product” refers to any one or more components of blood that can be delivered to a subject, for example, through a central venous catheter, for example a peripherally inserted central venous catheter (PICC, an embodiment of a central venous catheter as described herein). Red blood cells (RBCs), packed red blood cells (PRBCs), platelets, and fresh frozen plasma (FFP) are examples of such, without intending to be limiting. Transfusion of blood and blood products is well known in the art, and the skilled artisan would readily understand what is referred to by blood product in the context of the present disclosure.

The term “clinical well-being” as used herein, refers to a state or degree of clinical or physiological wellness or health of a patient. A clinician can evaluate a patient's clinical well-being by physical examination or performing one or more tests or assays.

The terms “administering,” “delivering,” and “introducing,” can be used interchangeably to indicate the introduction of a therapeutic composition or agent (e.g., compositions comprising one or more therapeutics as described herein) into the body of a subject. The therapeutic composition or agent can be administered through a device or system as described herein that results in the delivery of at least a portion of the composition or agent to a desired location in the subject such that the composition or agent retains its therapeutic capability. In embodiments, administration and/or delivery methods comprise intravenous delivery.

The term “administered continuously” refers to the continuous delivery of a therapeutic agent, e.g., compound, molecule, peptide, biologic, chemical, etc. over a continuous period of time, for example, a 0.5, 1, 2, 3, 4, 5, 6, 7, 8, 9, 10, 11, 12, 13, 14, 15, 16, 17, 18, 19, 20, 21, 22, 23 or 24-hour period.

As used herein, the terms “pharmaceutically acceptable” or “pharmacologically acceptable” refer to compositions that do not substantially produce adverse reactions, e.g., toxic, allergic, or immunological reactions, when administered to a subject.

The term “therapeutic agent” as used herein refers to one or more therapeutic substance selected from a group consisting of, but not limited to, one or more intravenous fluids, one or more vasoactive drugs, one or more antibiotics, one or more blood thinners, one or more sedating agents, one or more paralyzing agents, insulin, or one or more blood products in a pharmaceutically-acceptable fluidic vehicle, individually or in any combination of any thereof.

The terms “therapy,” “treatment,” and “amelioration” refer to any reduction in the severity of symptoms, e.g., of an adverse cardiac event (such as one that would require administration of a vasoactive drug or other therapeutics discussed herein), an adverse allergic event, low blood pressure, sepsis, a major surgery, a condition requiring total parenteral nutrition, a condition requiring intravenous antibiotics, immunosuppression, or a bone marrow transplant. As used herein, the terms “treat” and “prevent” are not intended to be absolute terms. Treatment can refer to any delay in onset, amelioration of symptoms, improvement in patient survival, improved cardiac function, improved blood pressure, improved nutrition, reduction in severity of an infection (or elimination thereof), offsetting immunosuppression, and increase in survival time or rate, etc. The effect of treatment can be compared to an individual or pool of individuals not receiving the treatment, or to the same patient prior to treatment or at a different time during treatment. In some aspects, the severity of disease or condition as discussed above is reduced by at least 5% or 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. The terms “treating” or “treatment” as used herein refers to an alleviation of symptoms associated with a disorder or disease or condition discussed above, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition. In particular, devices, systems, kits, and methods as described herein can be utilized to reduce or eliminate the chance of an infection following administration of a therapeutic as described herein for a condition as described herein utilizing administration methods that involve, for example, the use of tubing to deliver substances to the body of a subject (for example, a central line intravenous catheter).

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s). Pharmaceutical compositions of the present disclosure can comprise therapeutics as described herein that can be included in fluids that can be delivered to a patient.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “preventing” means to stop or hinder a disease, disorder, or symptom of a disease or condition through some action.

The term “reducing” means to diminish in extent, amount, or degree.

### Discussion

Described herein is technology comprising one or more ultraviolet “filter [s]” that can kill bacteria without harming drugs. Such filters (i.e., germicidal lights, such as UV-C emitting light emitting diodes (LEDs) can be placed, for example, on central venous catheters. In traditional healthcare settings, such lines and tubing (i.e., central venous catheters or intravenous catheters) can get infected and cause deadly infections, leading to tens of thousands of deaths per year and up $1.8 BB in healthcare costs annually in the US alone. Certain patient populations include subjects having, or suspected of having, diabetes, an adverse cardiac event, an adverse allergic event, low blood pressure, sepsis, a major surgery, a condition requiring total parenteral nutrition, a condition requiring re intravenous antibiotics, immunosuppression, or a bone marrow transplant. Diabetics are particularly susceptible to such infections cause by the introduction of bacteria through central line venous catheters.

Described herein are devices, systems, kits, and methods, for, among other things, irradiating or otherwise exposing fluids to UV-C irradiation. Such devices, systems, kits, and methods can be utilized for irradiating fluids and aspects of central lines that can be used to introduce therapeutics into a subject in order to reduce the potential for bacterial to enter a subject when administered fluids through, for example, a central line.

### I. STERILIZATION CARTRIDGES

Described herein are sterilization cartridges for exposing an aqueous therapeutic to UV light, in particular, UV-C light (i.e., light in the ultraviolet spectrum having a wavelength of about 100 to about 280 or about 285 nm or slightly higher). Sterilization cartridges as described herein can irradiate fluids to be introduced into a subject, thereby killing bacteria and reducing or otherwise eliminating the chance of the development of a central line-associated blood-stream infections (CLABSI).

### A. Embodiments of Sterilization Cartridges

In embodiments, a sterilization cartridge (also referred to herein as a fluid mixing cartridge), can comprise: a housing having a fluidic conduit, an inflow channel, and an outflow channel. The fluidic conduit, inflow channel, and outflow are in fluidic communication.

A side of the housing of the cartridge can be translucent and configured to expose the fluidic conduit to a light source. As the skilled artisan would understand, the translucent side can be totally translucent, or only translucent where facing the fluidic conduit. In embodiments, the translucent side of the housing comprises UV-translucent polymer or quartz glass. In embodiments, the UV-translucent polymer comprises TOPAS® 8007 X-10 polymer or other equivalents that allow for the passage of UV light, in particular, UV light of UV-C wavelengths described herein. The skilled artisan would further understand that the translucent side can be configured as to eliminate optical dead spots within the fluidic conduit to ensure that all of the fluid within the fluidic conduit is exposed to UV-C light in a sufficient amount to kill or eliminate bacteria that may be present in the fluid of the fluidic conduit. In embodiments, the remaining sides that are not configured to pass UV-C light to the fluidic conduit can be opaque and/or formed of a different material than the translucent side.

In embodiments, the housing of sterilization cartridges as described herein can optionally comprise an inflow adaptor in fluidic communication with the inflow channel. The inflow adaptor can be configured to removably couple to intravenous tubing. In embodiments, the housing of sterilization cartridges as described herein can optionally comprise an outflow adaptor in fluidic communication with the outflow channel. The outflow adapter can be configured to removably couple to intravenous tubing. In embodiments, the inflow adapter, the outflow adapter, or both, can comprise a Luer (ISO 80369-7) connection.

In embodiments, no adaptors are present that allow for the removable connection of tubings from the housing without a device such as scissors, a knife, or a scalpel that cuts the tubing from the housing. In embodiments, the inflow tubing, outflow tubing, or both can also be formed of a UV-translucent material, for example, the materials described herein.

In embodiments, the non-translucent sides of the housing (i.e., opaque sides) that do not transmit UV light can be formed of a UV-resistant polymer to aid in the reusing of the housing following exposure to UV light. Non-limiting examples of such UV-resistant polymers that can be used to form the non-translucent sides include acrylic, polycarbonate, high-density polyethylene, polyamid-imide, and polyvinylidene fluoride (PVDF)

In embodiments of sterilization cartridges described herein, the fluidic conduit can comprise a fluid mixing device. In embodiments, the fluid mixing device comprises one or more baffling structures in the fluidic conduit. The skilled artisan would understand that other arrangements of physical structures can be realized within the fluidic conduit to provide for mixing of components of the fluid to flow through the fluidic conduit.

In embodiments, in particular embodiments where fluids are administered manually by the provider without the aid of an infusion pump, for example, a sterilization cartridge can comprise a fluid restriction device in fluidic communication with the outflow channel. Examples of which include a three-way stopcock that can be altered by the user to reduce the aperture or radius of the tubing, or other devices known in the art that can decrease the radius of the cross-sectional area of the tubing to increase hydrodynamic resistance to flow.

In embodiments, the housing can further comprise one or more ultraviolet light (UV) sources. The one or more UV light sources can comprise one or more LEDs that emit light in the UV-C wavelength.

In embodiments, the housing can further comprise one or more flowmeters that can measure and/or regulate fluid flow through the sterilization cartridge. Non-limiting examples of which include a transit time flow probe, or thermistor flow sensor, for instance. In embodiments, a cartridge can comprise all of one or more ultraviolet light (UV) sources and a flowmeter.

In embodiments, cartridges can further comprise one or more indicator lights. In embodiments, the one or more indicator lights can be coupled to an exterior surface of the housing. In embodiments, the one or more indicator lights can comprise a photodiode. In embodiments, the one or more indicator lights can be configured to indicate the presence or lack or UV irradiation from the one or more light sources, the presence or lack of flow, insufficient UV irradiation from the one or more light sources, sufficient UV irradiation from the one or more light sources, and the like.

In embodiments, the housing of the sterilization can be configured to be reusable across the different users. In embodiments, the cartridge can be single use and disposable.

In embodiments, the housing of sterilization cartridges can comprise a power source. The power source can be from a battery or supplied by other devices as known ion the art. In embodiments, the power source comprises one or more insulated wires running axially through or attached to (for example, attached to an outer side) an intravenous tubing in fluidic connection with the inflow adaptor.

In embodiments, housings and other aspects of the present disclosure can be die cast using a mold using techniques known in the art. In embodiments, any one or more sides of the housing of the sterilization cartridge can be manufactured using three-dimensional printing techniques known it the art (also referred to as additive manufacturing). Such polymers that can be utilized in housings described herein can be any polymer or resin utilized in additive manufacturing so long as the finished product can withstand sterilization by aseptic techniques, such as UV irradiation, autoclaving, administration of alcohols, etc.

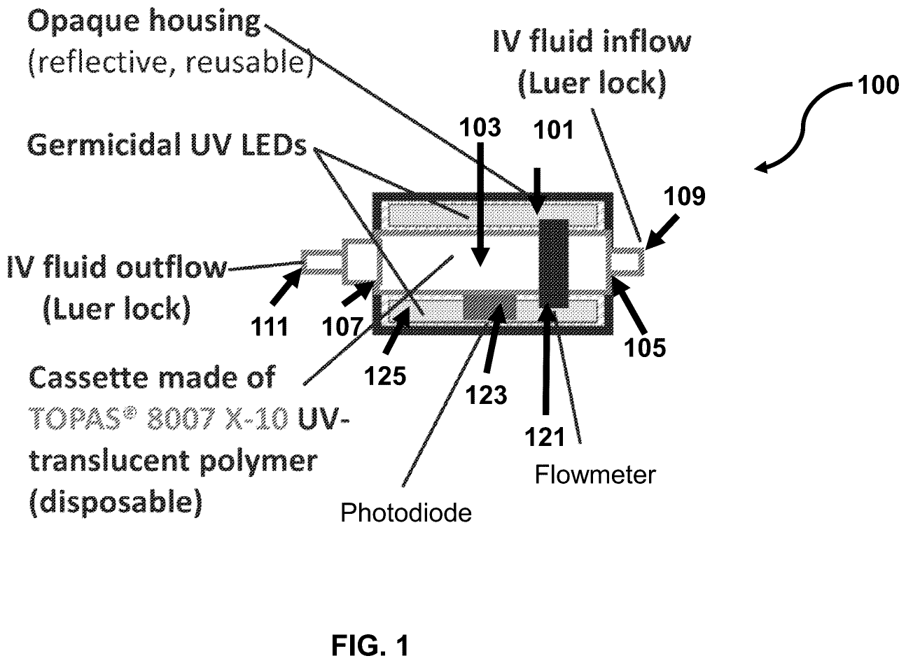

FIG. 1 depicts an embodiment of a sterilization cartridge 100 according to the present disclosure. As shown, a housing 101 has a fluidic conduit 103, an inflow channel 105, and an outflow channel 107. The fluidic conduit, inflow channel, and outflow are in fluidic communication. A side of the housing is translucent (side of the housing 101) that can be seen through, thereby configured to expose the fluidic conduit 103 to UV-C light. An inflow adaptor 109 is in fluidic communication with the inflow channel configured to removably couple to intravenous tubing; and an outflow adaptor 111 in fluidic communication with the outflow channel 107 configured to removably couple to intravenous tubing (not pictured).

The housing 101 can further comprise one or more ultraviolet light (UV) sources 125, a flowmeter 121, or a photodiode 123, according to various embodiments.

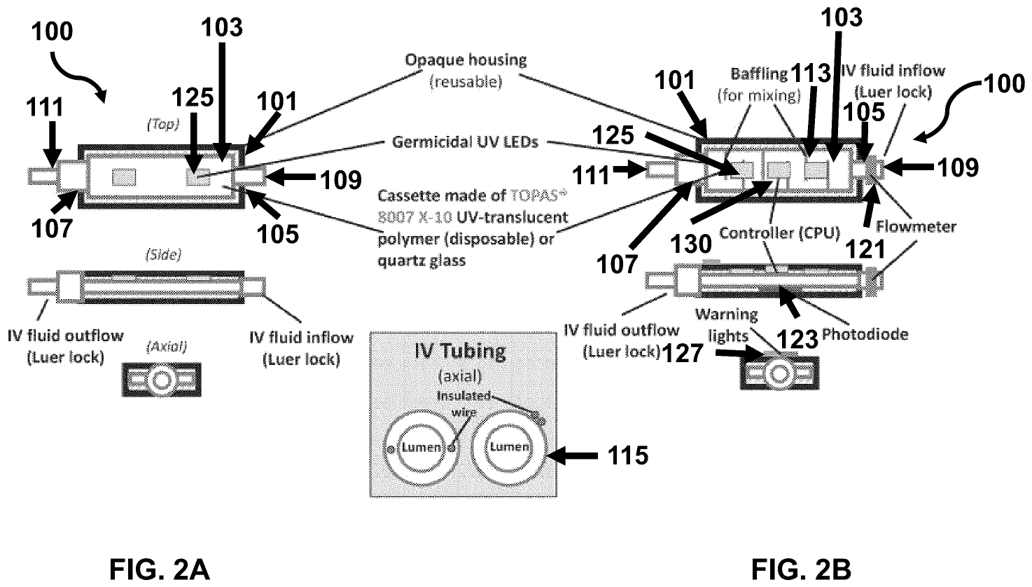

FIGS. 2A and 2B depict additional embodiments and aspects of sterilization cartridges 100 according to the present disclosure. As shown, a housing 101 has a fluidic conduit 103, an inflow channel 105, and an outflow channel 107. The fluidic conduit, inflow channel, and outflow are in fluidic communication. A side of the housing is translucent (side of the housing 101 that can be seen through, thereby configured to transmit UV-C from a light source, through the translucent side, thereby exposing the fluidic conduit 103 to UV-C light). An inflow adaptor 109 is in fluidic communication with the inflow channel configured to removably couple to intravenous tubing; and an outflow adaptor 111 in fluidic communication with the outflow channel 107 configured to removably couple to intravenous tubing 115 (pictured from an axial view).

The cartridge 100 can further comprise one or more fluid mixing devices, for example, the one or more baffling structures 113 in the fluidic conduit 103. In embodiments, cartridges can further comprise a fluid restriction device (not pictured) in fluidic communication with the outflow channel 107. The fluid restriction device (a check valve, for example) can be operably connected to the outflow adaptor 111 or the tubing (not pictured). The housing 101 can further comprise one or more ultraviolet light (UV) sources 125, a flowmeter 121, indicator lights 127, or a photodiode 123, according to various embodiments. In some embodiments, the housing 101 can further comprise one or more central processing units 130 configured to send and receive data from the flowmeter 121 and the one or more UV light sources 125, wherein the data sent to flowmeter 121, one or more UV light sources 125, or both, is based on a flow rate of a liquid through the cartridge 100 determined by the flowmeter 121.

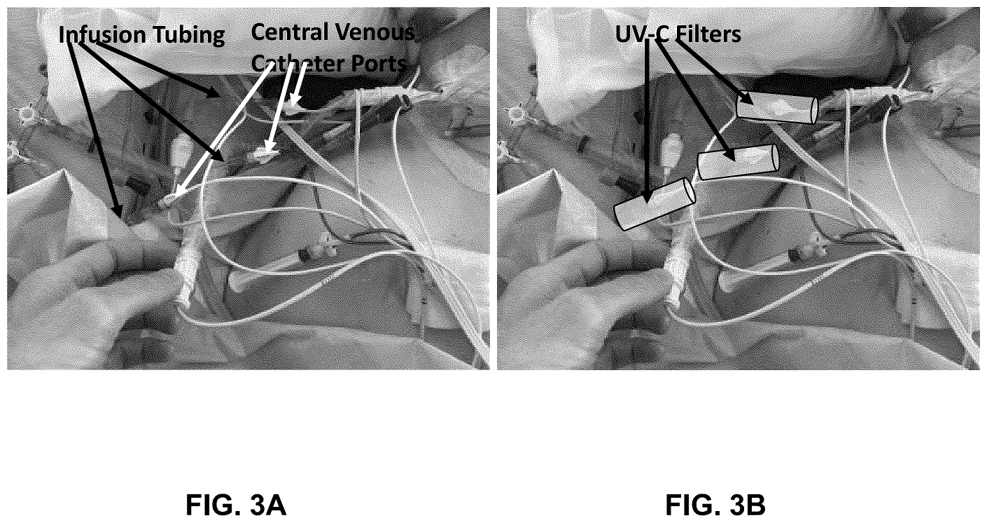

FIGS. 3A and 3B are photographs depicting an application of sterilization cartridges, systems, and methods of the present disclosure. FIG. 3A is a photograph showing infusion tubing and intravenous catheter ports of which cartridges, systems, kits, and methods of the present disclosure can be utilized with. FIG. 3B depicts embodiments of placement of sterilization cartridges according to the present disclosure in-line with the intravenous catheter ports through which fluids, in particular fluids containing therapeutics, can be delivered to a subject in need thereof.

### B. Central Processing Units

In embodiments, cartridges and systems as described herein can comprise one or more central processing units (CPUs). The one or more CPUs can be, for example, configured to send and receive data from the flowmeter, the one or more UV light sources, or both. The data sent to flowmeter, one or more UV light sources, or both, can be based on a flow rate of a liquid through the cartridge determined by the flowmeter. In embodiments, CPUs can be integrated in housings and/or systems according to the present disclosure. For example, in a system, the CPU may be within an infusion pump that is operably connected to housings and tubings described herein (i.e., configured to send and/or receive data to/from any one or more aspects of devices and systems described herein).

Described herein are systems for executing one of more or all of the method steps described herein that can be incorporated with or integrated with embodiments of devices, systems, and kits as described herein. Systems can comprise, for example, a machine readable medium, that, when executed, contains logic that executes any one or more of the steps of the method.

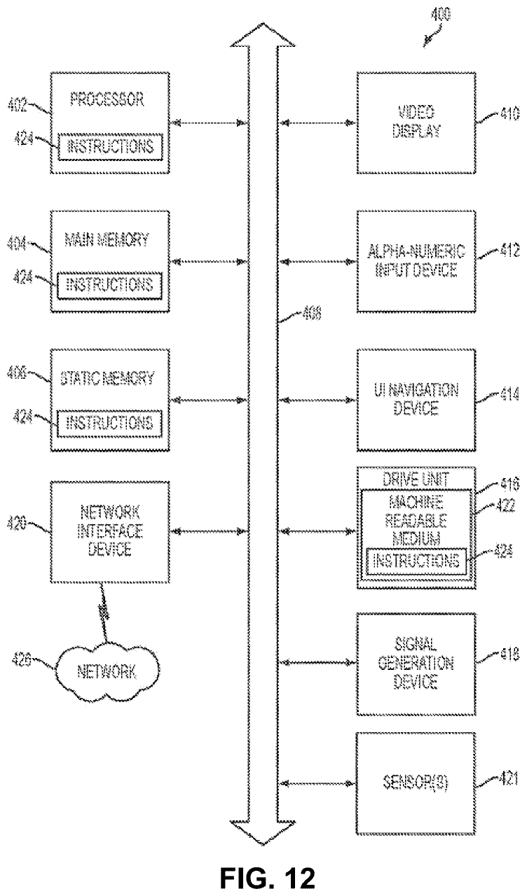

FIG. 12 is a block diagram illustrating an example of a machine upon which one or more aspects of embodiments of the present invention can be implemented (in particular, systems and machine-readable media that can perform the methods above).

Referring to FIG. 12, an aspect of an embodiment of the present invention includes, but not limited thereto, a device, system, method, and computer readable medium that provides for steps irradiating fluids to be introduced to a subject, which illustrates a block diagram of an example machine 400 upon which one or more embodiments (e.g., discussed methodologies) can be implemented (e.g., run).

Examples of a machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between suet, circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand- alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 414 (e.g., a mouse). In an example, the display unit 410, input device 412 and Ul navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one of, or any combination of, the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### II. SYSTEMS FOR FLUID STERILIZATION

Described herein are systems for fluid sterilization, in particular, sterilization of fluids to be administered to a subject through a central intravenous line. In embodiments, systems as described herein can comprise: a sterilization cartridge as described herein and one or more inflow or outflow tubings. In embodiments, systems can comprise one or more intravenous tubings configured to connect to an inflow adaptor; one or more intravenous tubings configured to connect to an outflow adaptor; and one or more ultraviolet light (UV) sources. In embodiments, tubings can be operably connected to the housing in a non-removable and permanent manner.

In embodiments, systems can further comprise a fluid restriction device in fluidic communication with the outflow channel when an infusion pump is not being used. Examples of which include a three-way stopcock that can be altered by the user to reduce the aperture or radius of the cross-sectional area of the tubing, or other devices known in the art that can decrease the radius of the cross-sectional area of the tubing to increase hydrodynamic resistance to flow.

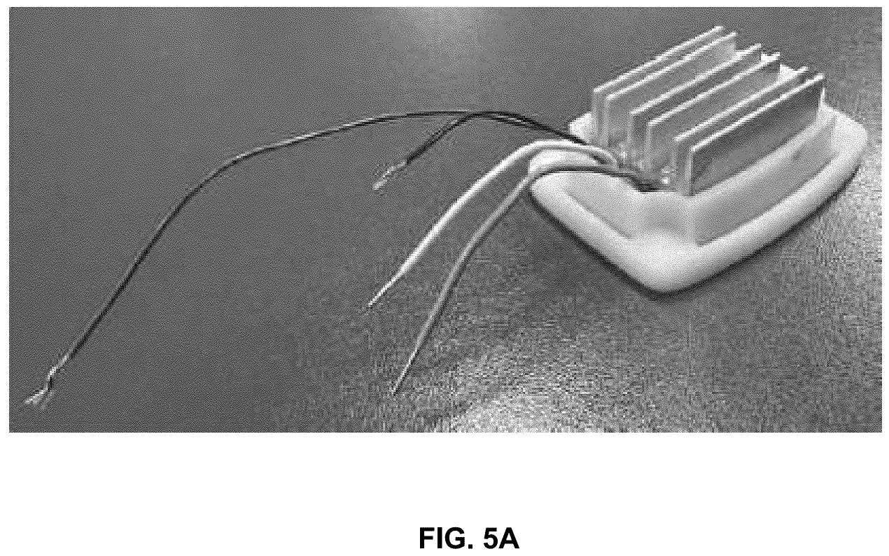

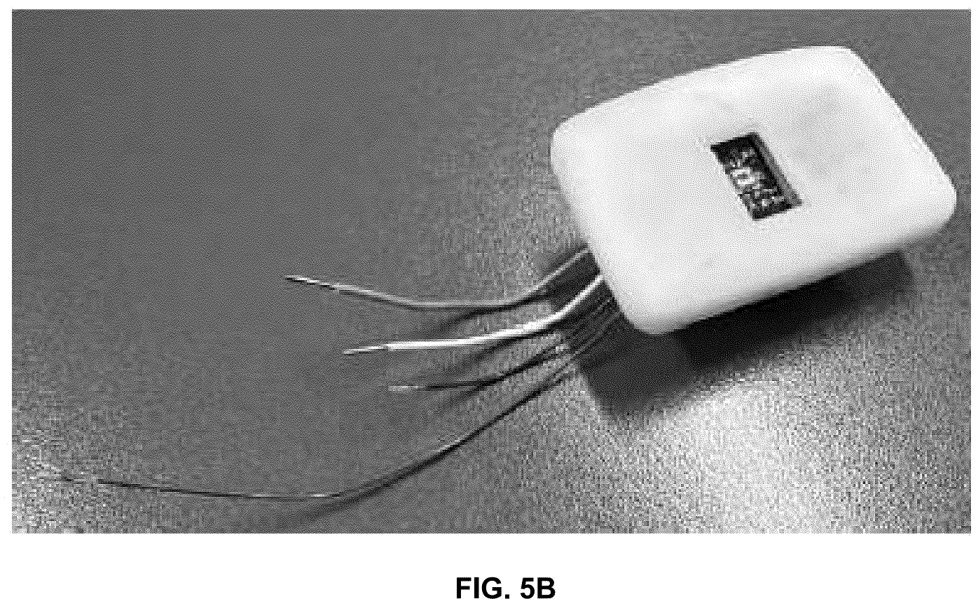

In embodiments of systems as described herein, the housing further comprises one or more baffling structures in the fluidic conduit. While embodiments are shown with a labyrinth or maze-like arrangement, the skilled artisan would understand that other configurations can be realized. In embodiments of systems as described herein, the translucent side of the housing comprises UV-translucent polymer or quartz glass. In embodiments of systems as described herein, the UV-translucent polymer comprises TOPAS® 8007 X-10 polymer. In embodiments of systems as described herein, systems further comprise a flowmeter configured to measure the fluid flow rate through the cartridge, the inflow channel, and/or the outlet channel. Systems can further comprise one or more heatsink for dissipation of heat generated by the power source, the photodiodes, or the UV-C light source (FIGS. 5A-5B).

In embodiments of systems as described herein, systems can further comprise one or more central processing units configured to send and receive data from the flowmeter and the one or more UV light sources, wherein the data sent to flowmeter, one or more UV light sources, or both, can be based on a flow rate of a liquid through the cartridge determined by the flowmeter. In embodiments of systems as described herein, systems can comprise one or more indicator lights configured to provide an indication output to the user regarding flow rate, UV-C irradiation, or any combination thereof. In embodiments of systems as described herein, the indicator light can comprise a photodiode.

In embodiments of systems as described herein, systems can further comprise a power source. In embodiments of systems as described herein, the power source can comprise a battery or one or more insulated wires running axially through at least one of the one or more intravenous tubings connected to the inflow adaptor.

In embodiments of systems as described herein, systems can further comprise one or more intravenous fluids, one or more vasoactive drugs, one or more antibiotics, one or more blood thinners, one or more sedating agents, one or more paralyzing agents, insulin, or one or more blood products in a pharmaceutically-acceptable fluidic vehicle. In embodiments of systems as described herein, the one or more vasoactive drugs can comprise insulin, epinephrine, Digoxin, Labetalol, or Amiodarone.

In embodiments of systems described herein, systems can further comprise an infusion pump that can be configured by the user to infuse or otherwise administer fluids through the housing and into a subject.

### III. KITS AND PACKAGING

Described herein are kits comprising one or more aspects of devices, systems, and methods of sterilization fluids for patient delivery. In embodiments, a kit can comprise a sterilization cartridge as described herein and one or more intravenous tubings. The one or more intravenous tubings can be permanently attached to the inflow and/or outflow channel of the housing. In embodiments, kits can comprise one or more intravenous tubings configured to connect to the inflow adaptor and be removable; and one or more intravenous tubings configured to connect to the outflow adaptor and be removable. In embodiments, one of the one or more intravenous tubings can comprise one or more insulated wires extending axially along an exterior surface or through walls of the intravenous tubing configured to provide power to the one or more ultraviolet light (UV) sources, the flowmeter, or both . . . or any other aspect of devices and systems described herein. Kits may also include one or more intravenous fluids as described herein or other therapeutics disclosed herein to be administered in an aqueous solution to a subject through a central line, for example.

A kit may additionally include other materials desirable from a commercial and user standpoint, including, without limitation, buffers, diluents, filters, needles, syringes, one or more therapeutics, and package inserts with instructions for performing any methods disclosed herein (e.g., methods for treating a disease disclosed herein). A therapeutic or formulation in a kit of the disclosure may comprise any of the formulations or compositions disclosed herein.

In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

A protocol as described in this disclosure for use in sterilizing fluids prior to delivery into a subject may be delivered in a pharmaceutical package or kit to doctors or other healthcare providers. Such packaging is intended to improve compliance with use instructions. Typically, the packaging comprises paper (cardboard) or plastic. In some embodiments, the kit or pharmaceutical package further comprises instructions for use (e.g., for administering according to a method as described herein).

### IV. METHODS OF USE AND TREATMENT

Described herein are methods of sterilizing fluids for delivery into a subject. Non-limiting examples of such fluids can be found in the present disclosure and can contain any one or more therapeutics as described herein (for example, blood products, vasoactive drugs, etc.).

In embodiments, a method of sterilizing fluids for delivery into a subject can comprise providing a fluidic path for delivery of a fluid to a subject by connecting the inflow channel and/or inflow adaptor of the sterilization cartridge as described herein to intravenous inflow tubing, connecting the outflow channel and/or outflow adaptor of the sterilization cartridge described herein to intravenous outflow tubing, and connecting the intravenous outflow tubing into a vein of the subject. Fluid can then be direct through the intravenous tubing into the sterilization cartridge, where ultraviolet (UV) light can be applied (transmitted through the translucent side) to the fluid as it passes through the fluidic conduit of the sterilization cartridge.

In embodiments, methods as described herein can comprise measuring an intensity of the UV light transmitted to the fluid with one or more photodiodes. In embodiments, methods can comprise measuring a flow rate with a flowmeter. In embodiments, methods can comprise adjusting the measured intensity of the UV light. In embodiments, this can be done based at least in part on the measured flow rate of the fluid through the intravenous tubing sterilization cartridge.

In embodiments of methods as described herein, the fluid can comprise one or more vasoactive drugs in a pharmaceutically-acceptable fluidic vehicle. In embodiments of methods as described herein, the one or more vasoactive drugs can comprise any one or more of insulin, epinephrine, Digoxin, Labetalol, or Amiodarone.

In embodiments, fluids can be delivered through the housing at flow rates as low as, for example, 5 mL/hr for an infusion of a single therapeutic agent, to 1000 mL/minute for a rapid infuser.

In embodiments of methods as described herein, following UV-C irradiation, the sterilized fluid is delivered to a subject that has, or is suspected of having, an adverse cardiac event (e.g., heart attack or stroke), an adverse allergic event (e.g., anaphylaxis), low blood pressure, sepsis, a major surgery (e.g., open-heart surgery), a condition requiring total parenteral nutrition (e.g., Crohn's disease), a condition requiring intravenous antibiotics (e.g., sepsis), immunosuppression (e.g., an organ transplant), or a bone marrow transplant.

In some embodiments, a composition or component of a composition described herein comprising a therapeutic described herein can be administered to a subject as a monotherapy. Alternatively, the composition or component of a composition described herein or therapeutic can be administered in conjunction with other therapies. For example, the composition can be administered to a subject at the same time, prior to, or after, a second therapy. In some embodiments, the composition or component of a composition described herein, and the one or more additional active agents are administered at the same time. Optionally, the composition or component of a composition described herein is administered first in time and the one or more additional active agents are administered second in time. In some embodiments, the one or more additional active agents are administered first in time and the therapeutic composition or component of a composition described herein is administered second in time. Optionally, the composition or component of a composition described herein, and the one or more additional agents are administered simultaneously in the same or different routes.

A composition as described herein can replace or augment a previously or currently administered therapy, such as previously prescribed blood pressure management medicament.

Monitoring a subject (e.g., a human patient) for development of symptoms of infection following administration of a sterilized fluid as described herein, means evaluating the subject for a change in a given parameter or symptom exhibited by the subject by a clinician in a social setting or self-reporting by the patient. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality. In the event an infection develops following central venous line administration of sterilized fluids, additional antibiotics can be administered.

In certain embodiments, the effective amount of a pharmaceutical composition comprising one or more therapeutics of the present disclosure to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a treatment is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the active components in the formulation used. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). In certain embodiments, a clinician administers the composition until a dosage is reached that achieves the desired effect. In certain embodiments, the therapeutic can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain examples, the compositions thereof can be irradiated and administered once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering a composition according to one of the aforementioned dosing regimens for a first period and another of the aforementioned dosing regimens for a second period. In some cases, the treatment discontinues for a period of time before the same or a different dosing regimen resumes. For example, a patient may be on a dosing regimen for two weeks, off for a week, on for another two weeks, and so on.

### i. Exemplary Treatments for Use with the Present Disclosure

The types of therapeutics, and some specific drugs, useful according to methods of the present disclosure include, but are not limited to: blood products, intravenous fluids (including isotonic, hypotonic, or hypertonic fluids); one or more vasoactive drugs (including insulin); one or more antibiotics; one or more blood thinners (e.g., warfarin); one or more sedating agents (e.g., propofol); one or more paralyzing agents (e.g., succinylcholine); insulin (as to help regulate blood pressure); or one or more blood products in a pharmaceutically-acceptable fluidic vehicle (e.g., blood components, including red blood cells, platelets, plasma, and cryoprecipitate).

Examples of intravenous fluids can include, without intending to be limiting: 0.9% normal saline (a common isotonic fluid that replaces electrolytes and fluids); dextrose 5% in 0.9% normal saline (D5NS) (a hypertonic fluid that treats hyponatremia and cerebral edema, for example); 3% sodium chloride (3% NaCl, a hypertonic fluid, for example, that treats hyponatremia and cerebral edema); 0.45% normal saline (a hypotonic fluid that moves fluid into cells to treat intracellular dehydration, for example); and 5% dextrose in water (D5W, a hypotonic fluid that treats hypernatremia and provides water for the kidneys to excrete solute, for example).

Examples of vasoactive drugs can include, without intending to be limiting, insulin, epinephrine, Digoxin, Labetalol, and Amiodarone.

The dosage of the active compound(s) being administered will depend on the condition being treated, the particular compound being administered, and other clinical factors such as age, sex, weight, and health of the subject being treated. It is to be understood that the present invention has application for both human and veterinary use. The drugs can be administered in formulations that contain all drugs being used, or the drugs can be administered separately. In some cases, it is anticipated that multiple doses/times of administration will be required or useful. The present invention further provides for varying the length of time of treatment.

### V. EXAMPLES

Now having described the embodiments of the disclosure, in general, the examples describe some additional embodiments. While embodiments of the present disclosure are described in connection with the examples and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1

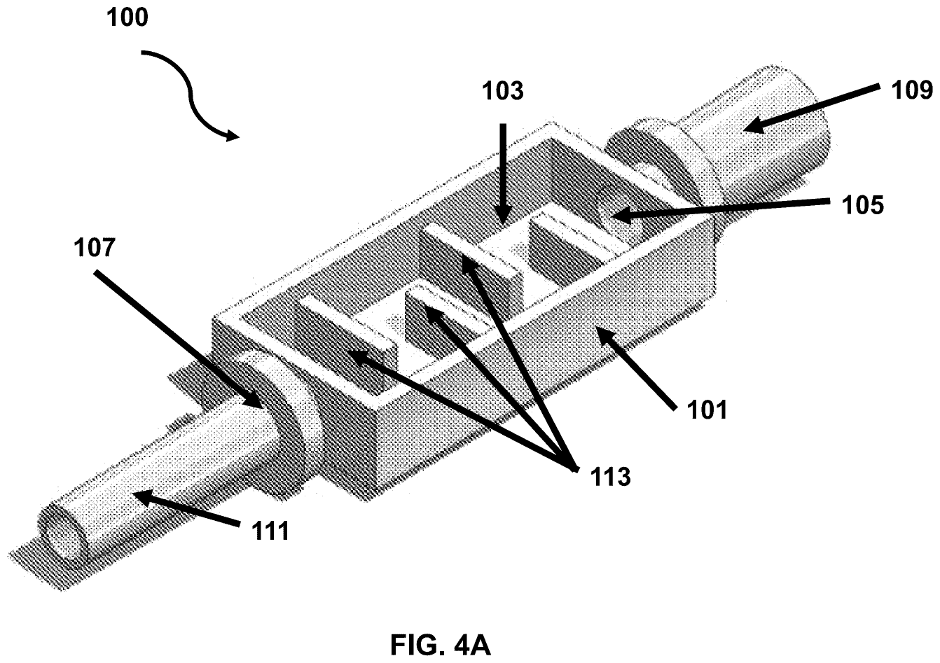

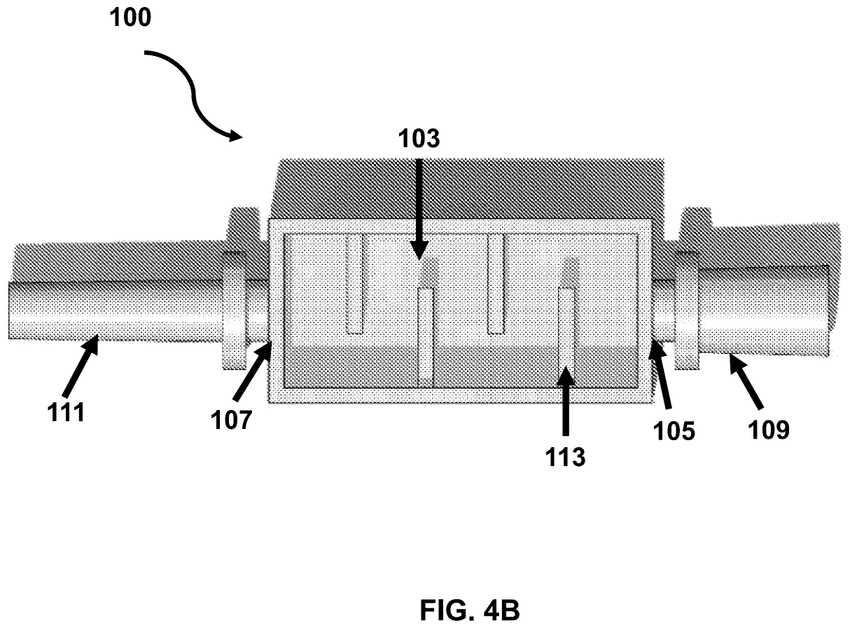

FIGS. 4A and 4B show an embodiment of a sterilization cartridge 100 according to the present disclosure. As shown, a housing 101 has a fluidic conduit 103, an inflow channel 105, and an outflow channel 107. The fluidic conduit, inflow channel, and outflow are in fluidic communication. A side of the housing is translucent (side of the housing 101 that can be seen through, thereby configured to transmit UV-C light from a light souce through the translucent side and expose the fluidic conduit 103 to UV-C light). An inflow adaptor 109 is in fluidic communication with the inflow channel configured to removably couple to intravenous tubing; and an outflow adaptor 111 in fluidic communication with the outflow channel 107 configured to removably couple to intravenous tubing (not pictured).

The cartridge 100 can further comprise one or more fluid mixing devices, for example, the one or more baffling structures 113 in the fluidic conduit 103. In embodiments, cartridges can further comprise a fluid restriction device (not pictured) in fluidic communication with the outflow channel 107. The fluid restriction device (a check valve, for example) can be operably connected to the outflow adaptor 111 or the tubing (not pictured).

### Example 2

FIGS. 5A and 5B show an embodiment of aspects of a system 200 according to the present disclosure. Depicted is an embodiment of a heat sink and germicidal LED (i.e., a UV-C emitting LED) that can be utilized with sterilization cartridges according to the present disclosure. FIGS. 5A and 5B are opposing surfaces of the same embodiment.

### Example 3

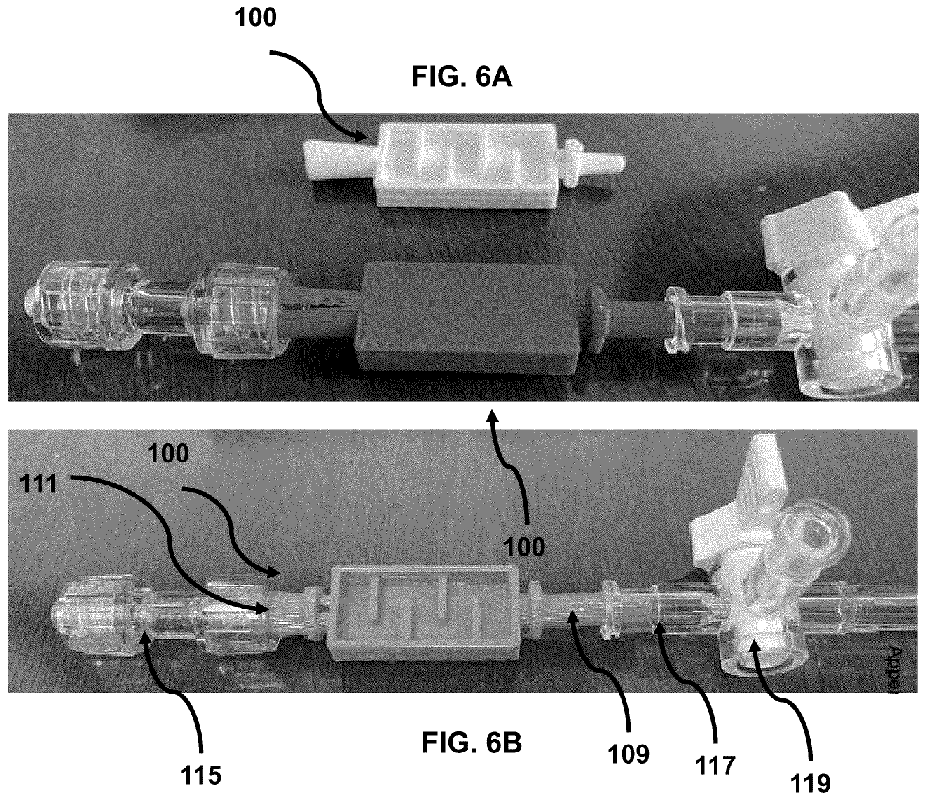

FIGS. 6A and 6B show embodiments of the sterilization cartridge 100 of FIGS. 4A and 4B with inflow and outflow adapters 109 and 111 that can be coupled to an aspect of tubing, intravenous or central line tubing, for example, 115 and 117. An embodiment of a fluid restriction device 119 is also shown (a valve).

### Example 4

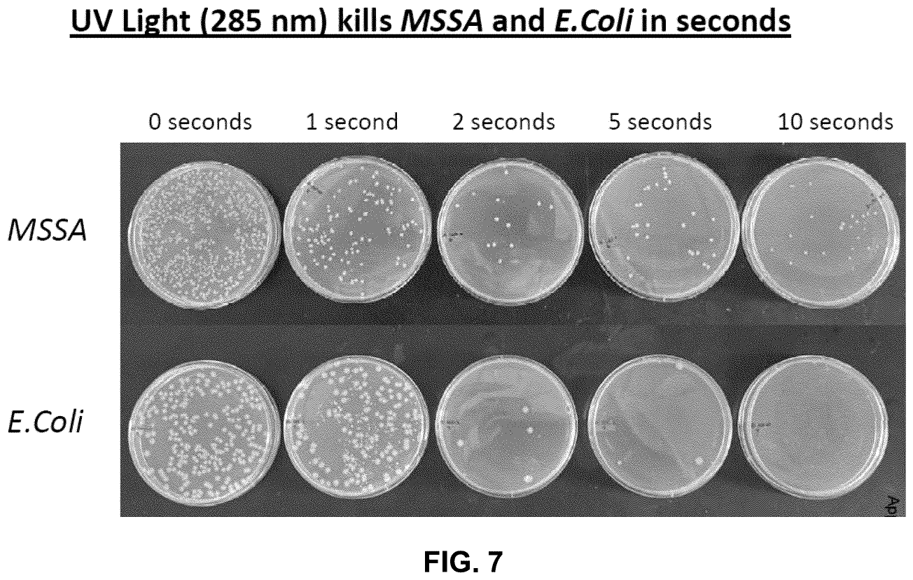

FIG. 7 shows the effect of exposure of UV light (285 nm) on bacterial cultures over time (0, 1, 2, 5, and 10 seconds), in particular Meticillin-Sensitive Staphylococcus aureus (MSSA) (top panels) and Escherichia coli (E. coli) according to aspects of the present disclosure.

### Example 5

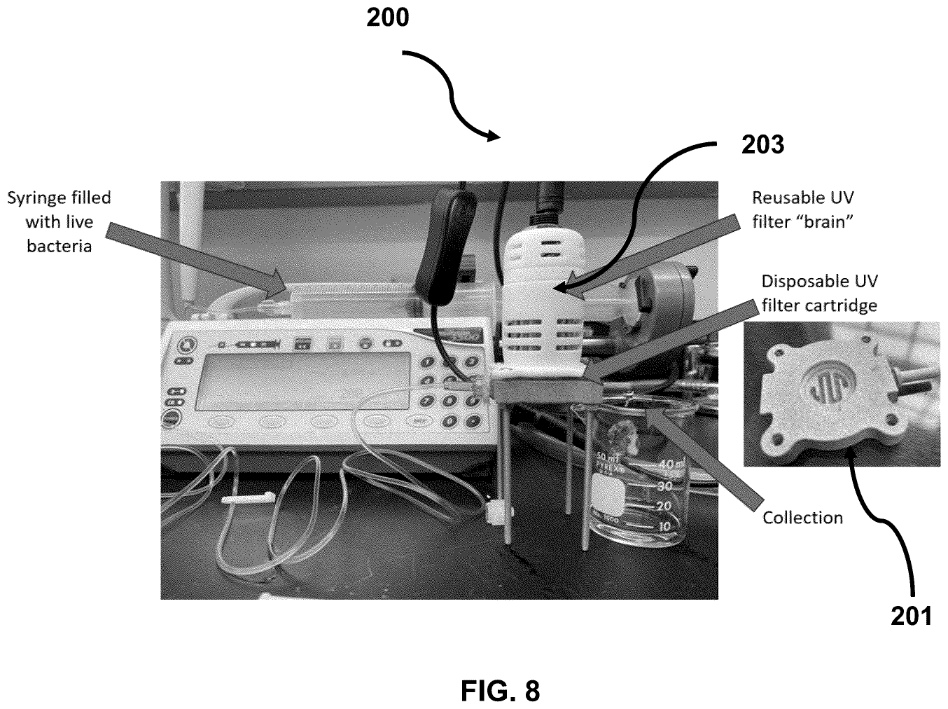

FIG. 8 shows an embodiment of a system 200 according to the present disclosure, comprising an embodiment of a sterilization cartridge 201 and UV light source 203 (also referred to herein as a UV filter). Other tubings and various aspects of the system for fluid flow are also shown. A TOPAS-8007 “lens” was glued onto the cartridge—this plastic is unique in that it is UV translucent and can be molded/mass produced using a die-cast part.

Several “UV sensitive” drugs (like epinephrine) were tested under the UV lamp at various time intervals with the system 200.

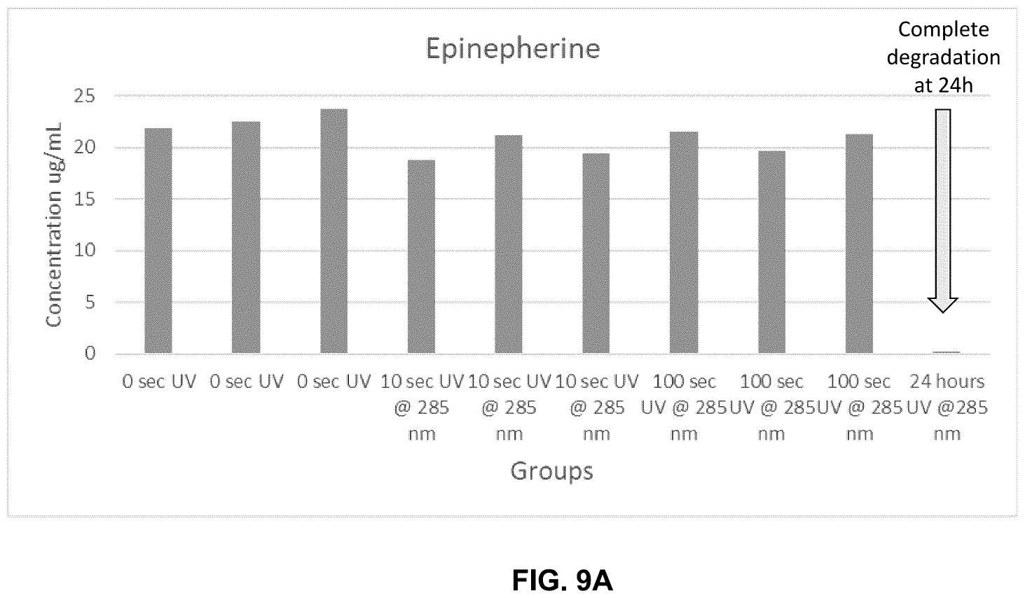

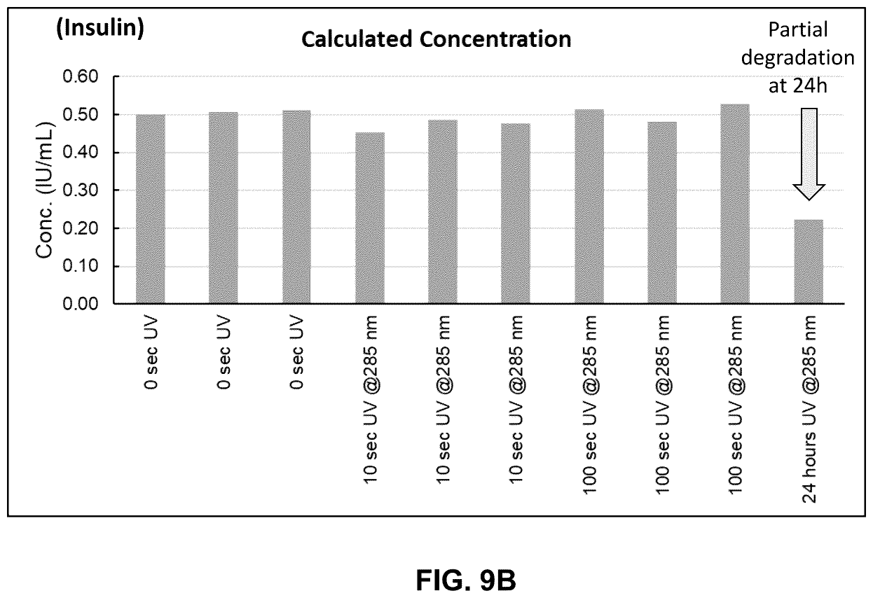

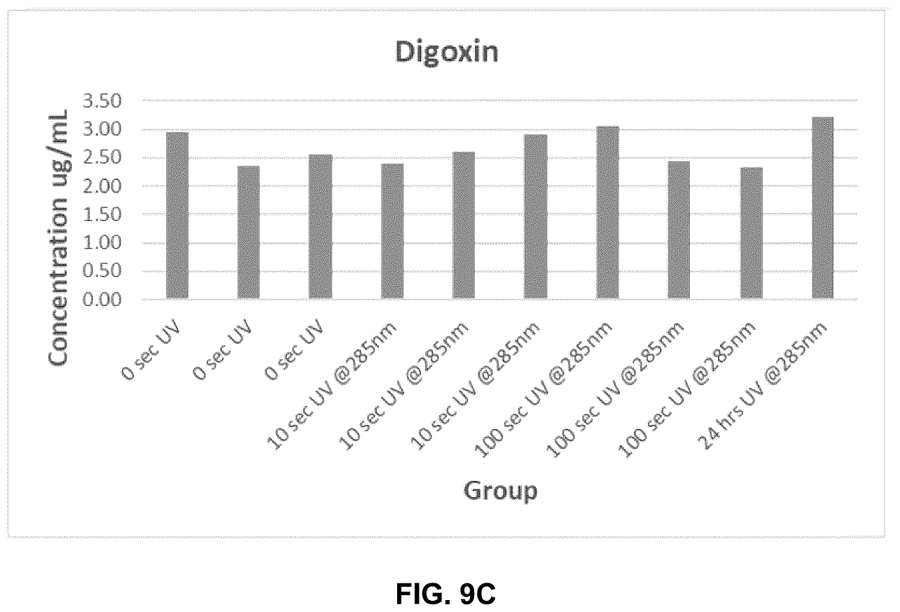

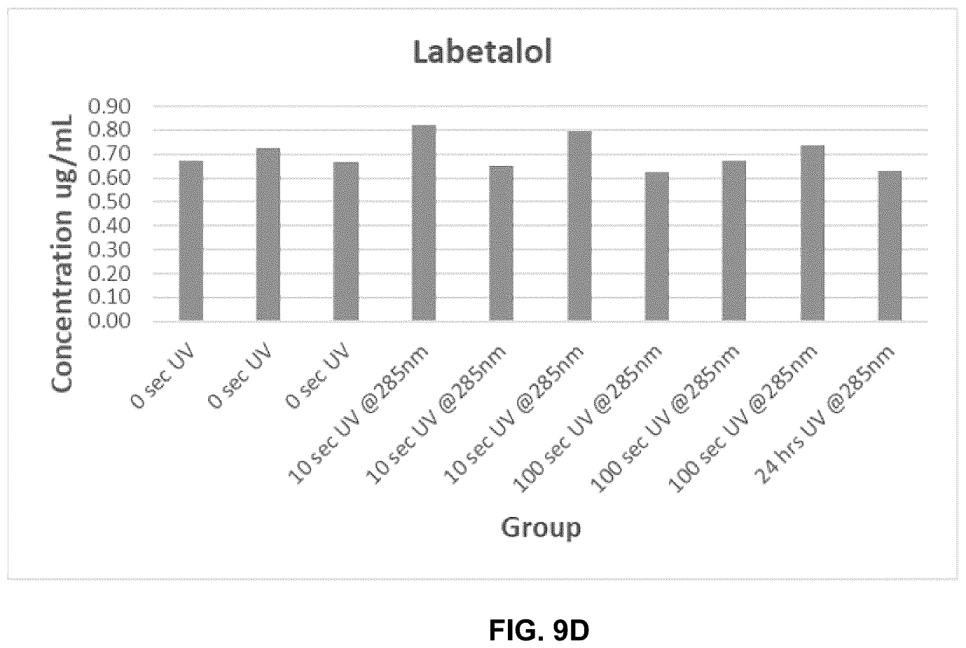

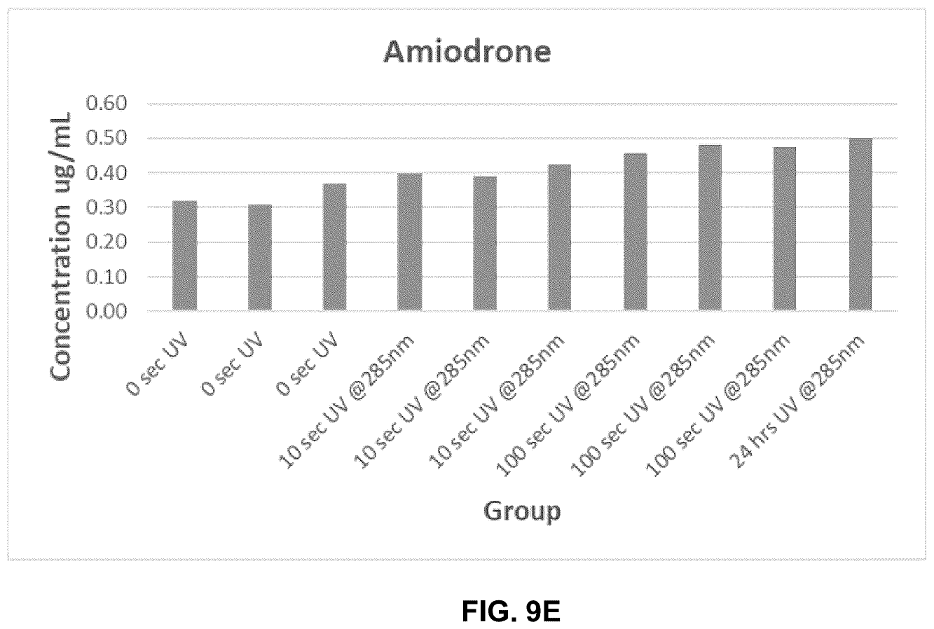

At a UV dose that is 10× what is needed to kill bacteria, it takes 24+ hours to degrade these drugs (FIGS. 9A-9E). In contrast, it takes 2 seconds to kill bacteria. That means that the UV light, at the doses used clinically, will not degrade the drugs infused into patients.

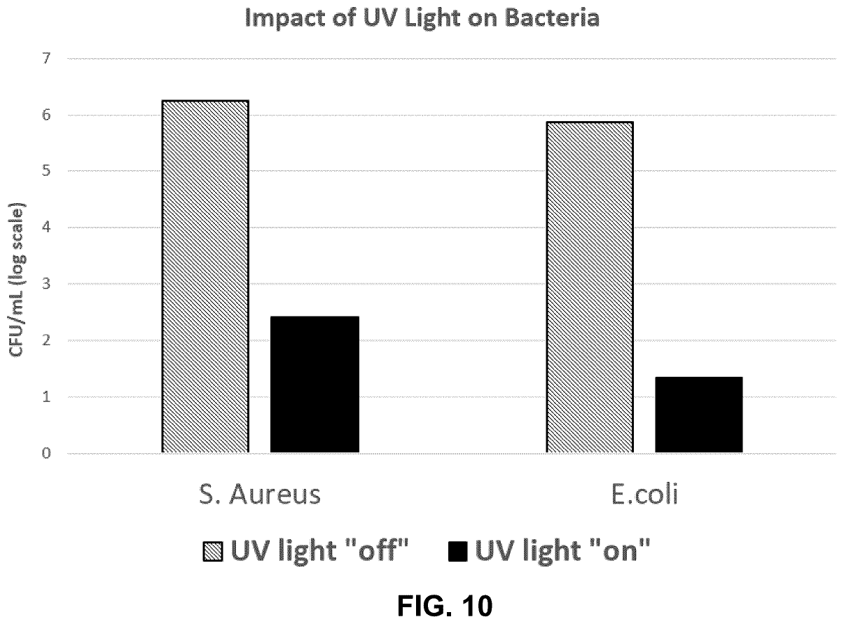

FIG. 10 is a plot showing the effect of UV-C irradiation on Staphylococcus aureus (S. aureas) and E. coli colony forming units. The initial CFU's tested were several log units greater than what may be practically present on a central line that could lead to CLABSI. A 4-log reduction was seen in bacteria following UV irradiation, running at a 200 mL fluid infusion flow rate, which is biologically relevant. As can be seen, the cartridge, the system, and the filter are effective at killing most of the bacteria.

### Example 6

Four (4) rats were exposed to 3e9 CFU of S.Aureus, injected through a tail vein. Four hours later, animals were euthanized, and the blood, spleen, liver, and kidney were harvested and cultured. The tail vein IV was also removed and cultured (similar to culturing a central line). For the first two rats, the bacteria were injected through the UV filter with the 285 nm LED on (UV group), for the second two rats, the LED was off so that the filter was inactive.

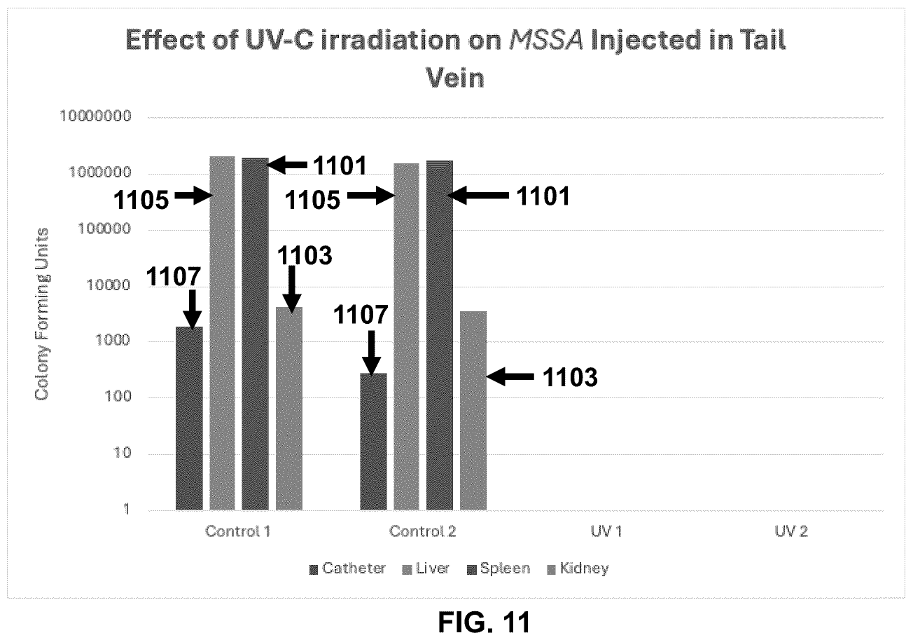

FIG. 11 shows the effect of UV-C irradiation on MSSA injected in the tail vein with and without UV-C irradiation. As can be seen, in non-irradiated lines, MSSA bacteria was found in the liver 1105, spleen 1101, and kidney 1103 of the animals, as well as the catheter 1107 itself, wherein with irradiated lines, no bacteria was found in the organs of the animals or the catheter.

Essentially, no bacteria were detected in any organ of the animals who received UV filtration, and bacteria was not detected on the tail vein catheter. In the two rats who did not get active UV filtration, bacteria were detected in all three organs as well as on the catheter.

### Example 7

The devices, systems, apparatuses, modules, compositions, computer program products, non-transitory computer readable medium, and methods of various embodiments of the present disclosure may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entireties (and which are not admitted to be prior art with respect to the present invention by inclusion in this section):
A. U.S. Utility patent application Ser. No. 13/780,207, entitled “System and Method for Magnetic Control ofan Anesthetic”, filed Feb. 28, 2013; Publication No. 2013/0225904, Aug. 29, 2013;
B. U.S. Utility patent application Ser. No. 14/435,230, entitled “OXIDATION MEASUREMENT SYSTEM AND RELATED METHOD THEREOF”, filed Apr. 13, 2015; U.S. Pat. No. 10,028,682, issued Jul. 24, 2018; Publication No. US-2015-0282747-AI, Oct. 8, 2015; and
C. International Patent Application Serial No. PCT/US2013/064737, entitled “OXIDATION MEASURENIENT SYSTEM AND RELATED METHOD THEREOF”, filed Oct. 12, 2013; Publication No. WO 2014/059399, Apr. 17, 2014.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A sterilization cartridge, comprising:
a housing having a fluidic conduit, an inflow channel, and an outflow channel, wherein the fluidic conduit, inflow channel, and outflow are in fluidic communication, and wherein a side of the housing is translucent and configured to expose the fluidic conduit to a UV-C light source.

2. The cartridge of claim 1, further comprising:
an inflow adaptor in fluidic communication with the inflow channel configured to removably couple to intravenous tubing; and
an outflow adaptor in fluidic communication with the outflow channel configured to removably couple to intravenous tubing.

3. The cartridge of claim 1, wherein the fluidic conduit comprises a fluid mixing device.

4. The cartridge of claim 1, wherein the translucent side of the housing comprises UV-translucent polymer or quartz glass.

5. The cartridge of claim 1, wherein the housing further comprises one or more ultraviolet light (UV) sources.

6. The cartridge of claim 5, wherein the housing further comprises a flowmeter.

7. The cartridge of claim 6, further comprising one or more central processing units configured to send and receive data from the flowmeter and the one or more UV light sources, wherein the data sent to flowmeter, one or more UV light sources, or both, is based on a flow rate of a liquid through the cartridge determined by the flowmeter.

8. The cartridge of claim 7, further comprising one or more indicator lights coupled to an exterior surface of the housing.

9. The cartridge of claim 1, wherein the housing further comprises a photodiode.

10. The cartridge of claim 1, further comprising a power source.

11. A kit, comprising:
the sterilization cartridge of claim 1; and
one or more intravenous tubings.

12. The kit of claim 11, wherein at least one of the one or more intravenous tubings comprises one or more insulated wires extending axially along an exterior surface or through walls of the intravenous tubing configured to provide power to the one or more ultraviolet light (UV) sources, the flowmeter, or both.

13. A system for sterilizing fluids in intravenous tubing, comprising:
the sterilization cartridge of claim 1;
one or more intravenous tubings configured to connect to the inflow adaptor;
one or more intravenous tubings configured to connect to the outflow adaptor; and
one or more ultraviolet light (UV) sources.

14. The system of claim 13 further comprising a flowmeter.

15. The system of claim 14, further comprising one or more central processing units configured to send and receive data from the flowmeter and the one or more UV light sources, wherein the data sent to flowmeter, one or more UV light sources, or both, is based on a flow rate of a liquid through the cartridge determined by the flowmeter.

16. The system of claim 15, further comprising one or more indicator lights coupled to an exterior surface of the housing.

17. The system of claim 16, wherein the housing further comprises a photodiode.

18. The system of claim 13, further comprising a power source.

19. The system of claim 13, further comprising one or more intravenous fluids, one or more vasoactive drugs, one or more antibiotics, one or more blood thinners, one or more sedating agents, one or more paralyzing agents, insulin, or one or more blood products in a pharmaceutically-acceptable fluidic vehicle.

20. A method of sterilizing fluids for delivery into a subject, comprising:
providing a fluidic path for delivery of a fluid to a subject by connecting the inflow adaptor of the sterilization cartridge of claim 1 to intravenous inflow tubing, connecting the outflow adaptor of the sterilization cartridge of claim 1 to intravenous outflow tubing, and connecting the intravenous outflow tubing into a vein of the subject;
directing a fluid through the intravenous tubing into the intravenous tubing sterilization cartridge; and
applying ultraviolet (UV) light to the fluid as it passes through the fluidic conduit of the sterilization cartridge.
